(19) United States
(12) Patent Application Publication
(10) Pub. No.: US 2018/0325958 A1
(43) Pub. Date: Nov. 15, 2018

(54) OSTEOGENIC GRAFT FORMING UNIT

(71) Applicant: OrthoCyte Corporation, Alameda, CA (US)

(72) Inventors: Francois Binette, Alameda, CA (US); Brent Atkinson, Highlands Ranch, CO (US); David Larocca, Alameda, CA (US)

(21) Appl. No.: 15/834,242

(22) Filed: Dec. 7, 2017

Related U.S. Application Data
(63) Continuation of application No. PCT/US2016/036778, filed on Jun. 9, 2016.
(60) Provisional application No. 62/172,808, filed on Jun. 9, 2015.

(51) Int. Cl. A61K 35/32; C12N 5/077; A61P 21/00; A61P 19/08

(52) U.S. Cl. CPC A61K 35/32; C12N 5/0655; C12N 5/0654; C12N 2533/80; A61P 19/08; C12N 2506/02; C12N 2533/54; A61P 21/00

(57) ABSTRACT

Disclosed herein are compositions comprising cell-derived preparations and/or bioactive substances derived therefrom, in combination with biological carriers. Methods for making the aforementioned compositions, and methods for their use in stimulating osteogenesis and chondrogenesis in subjects in need thereof, are also disclosed.

### PRIORITY

This application is a continuation of International Patent Application No. PCT/US16/36778 filed on Jun. 9, 2016 which claims priority to U.S. Provisional Patent Application No. 62/172,808, filed on Jun. 9, 2015. The entire contents of each of which are hereby incorporated by reference.

### FIELD

The present disclosure relates to osteogenic and chondrogenic precursor cells, and compositions comprising said precursor cells that promote osteogenesis and bone repair.

### BACKGROUND

Allogeneic bone grafts (e.g., demineralized bone matrix or DBM) are commonly utilized in orthopedic procedures. When bone is demineralized, endogenous osteogenic factors such as, for example, bone morphogenetic proteins (BMPs) become available for osteoinduction when implanted into a recipient. DBM is generally obtained from cadaveric donors; hundreds to thousands of donors are required for manufacture of commercial lots. The human donors used for the manufacture of DBM are quite variable in age, health status, quality of bone, amount of growth factors, etc., which leads to substantial variability from lot-to-lot.

More recently-developed bone allograft compositions contain both DBM and live cells. These products contain, in addition to DBM, cancellous bone, which contains both precursor cells and lineage-committed cells. Processing of such grafts removes the immunogenic cells of the bone marrow, but retains viable cells that are not immunogenic. However, the effectiveness of these compositions is limited by the dose of cells that can be provided. Furthermore, although these compositions contain physiological levels of cells, few stem cells are present in these preparations. Moreover, because they contain live cells, they have a limited shelf-life, challenging transport requirements and they cannot be sterilized, thus posing a risk of transmitting infection after transplantation. In addition, since they, too, are derived from a wide range of human donors, they also suffer from substantial lot-to-lot variability.

Additional existing methods for promoting bone formation comprise preparations that contain non-physiological (e.g., supraphysiological) levels of recombinant human bone morphogenetic protein-2 (BMP-2). Although high doses can be provided with these compositions, they provide only a single osteoinductive protein supplied at non-physiological levels, thus distorting biological homeostasis. An additional concern with the use of such preparations is the possibility of ectopic bone formation resulting from diffusion or migration of the recombinant protein from the transplant site. For example, if implanted BMP-2 migrates outside of the vertebral body during spinal fusion, bone can form and impinge on the nerves, which can result in patient pain. Supraphysiological levels of BMP-2 can also cause an inflammatory response, which can lead to severe dysphagia after cervical fusion, and possibly death.

Accordingly, new methods and compositions for bone grafting are needed. Such methods and compositions should:

(1) provide naturally-occurring mixtures of osteoinductive and/or osteopromotive factors, ideally present at physiological ratios;

(2) not be subject to extreme lot-to-lot variability with respect to the mixtures and concentrations described in (1);

(3) be rich in progenitor and/or precursor cells and/or their bioactive substances;

(4) have an extended shelf life;

(5) be easy to store and transport; and/or

(6) be amenable to sterilization.

The invention described in the present disclosure fulfills these needs and additional needs in the field.

### SUMMARY

In various embodiments described herein, the present disclosure provides compositions useful for stimulating bone formation (e.g., compositions that are osteoinductive and/or osteopromotive) in a subject, wherein, in certain embodiments, the compositions comprise a cell-derived preparation obtained from osteogenic precursor cells combined with a biological carrier. In certain embodiments, the osteogenic precursor cells are obtained by in vitro differentiation of osteogenic progenitor cells. Exemplary osteogenic progenitor cells include the SM30, MEL2 and SK11 cell lines. Exemplary cell-derived preparations include lysates, extracts, lyophilisates, exosome preparations and conditioned medium. Exemplary biological carriers include collagen (e.g., collagen sponges) and hydrogels.

In additional embodiments, the compositions comprise one or more bioactive substances (e.g., osteoinductive or osteopromotive substance) combined with a biological carrier. Sources of bioactive substances include, but are not limited to, cell lysates, cell extracts, exosomes, and conditioned medium from osteogenic precursor cells; as well as purified osteoinductive and/or osteopromotive proteins.

Also provided are methods for making the disclosed compositions, wherein the methods comprise combining osteogenic precursor cells, and/or a cell-derived preparation obtained from osteogenic precursor cells, and/or one or more bioactive substances with a biological carrier. In certain embodiments, the method comprises obtaining osteogenic precursor cells, optionally differentiating the osteogenic precursor cells by culturing the cells in the presence of one or more suitable differentiation factors, and applying the osteogenic precursor cells and/or differentiated cells to a biological carrier. In certain embodiments the osteogenic precursor cells and/or their differentiated progeny are subjected to a purification or an enrichment step before they are applied to the biological carrier. In other embodiments, the osteogenic precursor cells and/or their differentiated progeny are processed to obtain a cell-derived preparation that is applied to the biological carrier. Exemplary cell-derived preparations include lysates, extracts, exosome preparations and conditioned medium. In some embodiments, the biological carrier and the cells or the cell-derived preparations are processed to obtain a graft that can be stored for an extended period of time. An exemplary processing method is lyophilization (i.e., freeze-drying) of a cell-seeded biological carrier.

In certain embodiments, the method comprises co-culturing osteogenic precursor cells or their differentiated progeny with the biological carrier, such that the cells attach to the carrier, and subsequently removing the cell-seeded carrier from the culture. In some embodiments, the biological carrier and the cells are subsequently processed to obtain a graft that can be stored for an extended period of time. An exemplary method of processing is lyophilization (i.e., freeze-drying) of a cell-seeded biological carrier.

Bioactive substances (e.g., purified proteins, lysates, extracts, conditioned medium, exosomes) can be applied directly to a biological carrier. Alternatively, for cell lysates, a biological carrier can be co-cultured with cells, and the cells then lysed such that cellular contents remain adsorbed to the carrier. Following the combining step, biological carriers seeded with bioactive substances (such as, for example, purified proteins, lysates, extracts, conditioned medium, exosomes) can optionally be processed (e.g., lyophilized) to obtain a graft that can be stored for an extended period of time. Also provided are methods for stimulating bone formation in a human or animal subject, wherein the methods comprise transplanting the compositions described herein to a site in the subject at which bone formation is desired.

Although the instant compositions can be used allogeneically, they are different from previous allogeneic compositions in that the instant disclosure enables use of large numbers (e.g., ˜1 million) of clonally derived precursor cells (i.e. precursor cells derived from a clonal embryonic progenitor cell line) which are cultured in vitro and processed to collect osteogenic compositions. The osteogenic compositions derived from the clonally derived precursor cells are then added to synthetic bone void fillers. Since all lots of the product can be manufactured from a single clonal cell line (i.e., a single donor), less lot-to-lot variability will ensue, compared to existing products such as DBM or live-cell-containing bone grafts.

In addition, the graft-forming units disclosed herein provide mixtures of osteogenic, osteoinductive and/or osteopromotive molecules (e.g., growth factors and cytokines) at physiological ratios with respect to one another. Compared to existing methods, the instant methods, which provide physiological ratios of a combination of proteins, are unlikely to cause severe adverse effects such as ectopic ossification and inflammatory responses.

Finally, the compositions disclosed herein provide off-the-shelf products that can be sterilized (minimizing the risks of transmitting infection upon transplantation) and stored either refrigerated or at room temperature.

Accordingly, the present disclosure provides, inter alia, the following embodiments.

1. A composition comprising:
(a) a cell-derived preparation from an osteogenic precursor cell, and
(b) a biological carrier;

wherein the osteogenic precursor cell is not a mesenchymal stem cell.

2. The composition of embodiment 1, wherein the cell-derived preparation is selected from the group consisting of one or more of
(a) a lyophilisate of an osteogenic precursor cell;
(b) a lysate of an osteogenic precursor cell;
(c) an extract of an osteogenic precursor cell
(d) an exosome suspension from an osteogenic precursor cell; and
(e) conditioned medium from an osteogenic precursor cell.

3. The composition of either of embodiments 1 or 2, wherein the osteogenic precursor cell is obtained by differentiation of a progenitor cell.

4. The composition of embodiment 3, wherein the progenitor cell is a clonal embryonic progenitor cell.

5. The composition of either of embodiments 1 or 2, wherein the osteogenic precursor cell is obtained by differentiation of a clonal progenitor cell line selected from the group consisting of a SM30, MEL2 and SK11.

6. The composition of embodiment 5, wherein the osteogenic precursor cell is obtained by culturing a progenitor cell in the presence of TGF-β3, BMP-2, or both.

7. The composition of any of embodiments 3-6, wherein the progenitor cell expresses one or more of the following markers: MMP1, MYL4, ZIC2, DIO2, DLK1, HAND2, SOX11, COL21A1, PTPRN and ZIC1.

8. The composition of any of embodiments 1-7 wherein the osteogenic precursor cells express one or more markers chosen from integrin-binding sialoprotein (TBSP), osteopontin (SPP1), alkaline phosphatase, tissue-nonspecific isozyme (ALPL), and BMP-2.

9. The composition of any of embodiments 1-8, wherein the osteogenic precursor cell is a human cell.

10. The composition of any of embodiments 1-9, wherein the osteogenic precursor cell is not part of an embryoid body.

11. The composition of any of embodiments 1-10, wherein the osteogenic precursor cell is a member of a clonal cell population.

12. The composition of any of embodiments 1-11, further comprising a cell-derived preparation from a chondrogenic precursor cell, wherein the chondrogenic precursor cell is obtained by differentiation of a progenitor cell.

13. The composition of embodiment 12, wherein the cell-derived preparation is selected from the group consisting of one or more of

(a) a lyophilisate of a chondrogenic precursor cell;

(b) a lysate of a chondrogenic precursor cell;

(c) an extract of a chondrogenic precursor cell

(d) an exosome suspension from a chondrogenic precursor cell; and

(e) conditioned medium from a chondrogenic precursor cell.

14. The composition of either of embodiments 12 or 13, wherein the chondrogenic precursor cell is obtained by differentiation of a clonal progenitor cell line selected from the group consisting of 4D20.8, 7PEND24, 7SMOO32 and E15.

15. The composition of any of embodiments 12-14, wherein the progenitor cell expresses one or more of the following markers: DIO2, DLK1, FOXF1, GABRB1, COL21A1, and SRCRB4D.

16. The composition of any of embodiments 12-15, wherein the chondrogenic precursor cell expresses one or more markers chosen from collagen, type II, alpha 1 (COL2A1) and aggrecan (ACAN).

17. The composition of any of embodiments 1-16, wherein the biological carrier is a collagen, a collagen coated with a ceramic, a hydrogel, or a hydrogel supplemented with a ceramic.

18. The composition of any of embodiments 1-16, wherein the biological carrier is not demineralized bone matrix (DBM).

19. The composition of any of embodiments 1-18, wherein the composition is sterilized.

20. A method for promoting formation of bone and/or cartilage in a subject, the method comprising transplanting, into the subject, the composition of any of embodiments 1-19.

21. The method of embodiment 20, wherein the subject is a human.

22. The method of embodiment 20, wherein the subject is a non-human animal.

23. A method for making a therapeutic composition for promoting bone formation, the method comprising:

(a) growing progenitor cells in culture;

(b) differentiating the progenitor cells to osteogenic precursor cells (OPCs) in the culture;

(c) combining the OPCs with a biological carrier; and

(d) lyophilizing the combination of step (c).

24. A method of making a therapeutic composition for promoting bone formation, the method comprising:

(a) growing progenitor cells in culture;

(b) differentiating the progenitor cells to osteogenic precursor cells (OPCs) in the culture;

(c) combining the OPCs with a biological carrier; and

(d) lysing the cells present on the biological carrier to generate a graft-forming unit.

25. A method of making a therapeutic composition for promoting bone formation, the method comprising:

(a) growing progenitor cells in culture;

(b) differentiating the progenitor cells to osteogenic precursor cells (OPCs) in the culture;

(c) obtaining a lysate of the OPCs; and

(d) combining the lysate with a biological carrier to generate a graft-forming unit.

26. A method of making a therapeutic composition for promoting bone formation, the method comprising:

(a) growing progenitor cells in culture;

(b) differentiating the progenitor cells to osteogenic precursor cells (OPCs) in the culture;

(c) obtaining an extract from the OPCs; and

(d) combining the extract with a biological carrier to generate a graft-forming unit.

27. A method of making a therapeutic composition for promoting bone formation, the method comprising:

(a) growing progenitor cells in culture;

(b) differentiating the progenitor cells to osteogenic precursor cells (OPCs) in the culture;

(c) preparing exosomes from the OPCs; and

(d) combining the exosomes with a biological carrier to generate a graft-forming unit.

28. A method of making a therapeutic composition for promoting bone formation, the method comprising:

(a) growing progenitor cells in culture;

(b) differentiating the progenitor cells to osteogenic precursor cells (OPCs) in the culture;

(c) obtaining conditioned medium from the culture; and

(d) combining the conditioned medium with a biological carrier to generate a graft-forming unit.

29. The method of any of embodiments 24-28, the method further comprising, subsequent to step (d):

(e) lyophilizing the graft-forming unit of step (d).

30. The method of any of embodiments 23-29, wherein the progenitor cells are clonal embryonic progenitor cells.

31. The method of any of embodiments 23-30, wherein the progenitor cells are selected from the group consisting of SM30, MEL2 and SK11 cell lines.

32. The method of any of embodiments 23-31, wherein the progenitor cell expresses one or more of the following markers: MMP1, MYL4, ZIC2, DIO2, DLK1, HAND2, SOX11, COL21A1, PTPRN and ZIC1.

33. The method of any of embodiments 23-32, wherein the progenitor cells are differentiated to OPCs by culturing the progenitor cells in the presence of TGF-β3, BMP-2, or both.

34. The method of any of embodiments 23-33, wherein the OPCs express one or more markers chosen from bone sialoprotein II (TBSP), osteopontin (SPP1) and alkaline phosphatase, tissue-nonspecific isozyme (ALPL).

35. The method of any of embodiments 23-34, wherein the OPCs are human cells.

36. The method of any of embodiments 23-35, wherein the culture of OPCs does not comprise embryoid bodies.

37. The method of any of embodiments 23-36, wherein the culture of OPCs is a clonal culture.

38. The method of any of embodiments 23-37, wherein the biological carrier is a collagen, a collagen coated with a ceramic, a hydrogel, or a hydrogel supplemented with a ceramic.

39. The method of embodiment 38, wherein the collagen is gelatin.

40. The method of any of embodiments 23-39, wherein the biological carrier is not demineralized bone matrix (DBM).

41. A composition comprising:
(a) a cell-derived preparation from a chondrogenic precursor cell, and
(b) a biological carrier;

wherein the chondrogenic precursor cell is not a mesenchymal stem cell.

42. The composition of embodiment 41, wherein the cell-derived preparation is selected from the group consisting of one or more of
(a) a lyophilisate of a chondrogenic precursor cell;
(b) a lysate of a chondrogenic precursor cell;
(c) an extract of a chondrogenic precursor cell;
(d) an exosome suspension from a chondrogenic precursor cell; and
(e) conditioned medium from a chondrogenic precursor cell.

43. The composition of either of embodiments 41 or 42, wherein the chondrogenic precursor cell is obtained by differentiation of a progenitor cell.

44. The composition of embodiment 43, wherein the progenitor cell is a clonal embryonic progenitor cell.

45. The composition of either of embodiments 41 or 42, wherein the chondrogenic precursor cell is obtained by differentiation of a clonal progenitor cell line selected from the group consisting of 4D20.8, 7PEND24, 7SMOO32 and E15.

46. The composition of embodiment 45, wherein the chondrogenic precursor cell is obtained by culturing a progenitor cell in the presence of TGF-β3, GDF5, BMP-4, or combinations thereof.

47. The composition of any of embodiments 43-46, wherein the progenitor cell expresses one or more of the following markers: DIO2, DLK1, FOXF1, GABRB1, COL21A1, and SRCB4D.

48. The composition of any of embodiments 41-47 wherein the chondrogenic precursor cells express one or more markers chosen from COL2A1 and ACAN.

49. The composition of any of embodiments 41-48, wherein the chondrogenic precursor cell is a human cell.

50. The composition of any of embodiments 41-49, wherein the chondrogenic precursor cell is not part of an embryoid body.

51. The composition of any of embodiments 41-50, wherein the chondrogenic precursor cell is a member of a clonal cell population.

### BRIEF DESCRIPTION OF THE DRAWINGS

FIG. 1 shows thin sections, stained with Masson's Trichrome, of cell-seeded collagen sponges implanted into rats, six weeks after implantation. “Sponge control” refers to implants containing only collagen sponge. “SM-30” refers to implants containing collagen sponge seeded with SM30 cells. “Mel2” refers to implants containing collagen sponge seeded with MEL2 cells. “BMP-2” refers to implants containing collagen sponge seeded with 0.3 μg/μL of bone morphogenetic protein-2. The “Sponge control”, “SM-30” and “Mel2” sponges were lyophilized prior to implantation.

### DETAILED DESCRIPTION

The present disclosure employs, unless otherwise indicated, standard methods and conventional techniques in the fields of cell biology, molecular biology, embryology, biochemistry, cell culture, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N J, 2000; and the series “Methods in Enzymology,” Academic Press, San Diego, Calif.

For the purposes of the present disclosure, a “progenitor cell” is a pluripotent cell which can be induced, in vivo or in vitro, to differentiate into a cell that has a more restricted differentiation potential. Exemplary progenitor cells include the SM30, MEL2 and SK11 osteogenic cell lines.

The term “precursor cell,” as used herein, is a cell that is not pluripotent and is not terminally differentiated, but which is capable of differentiating into a terminally differentiated cell. Thus, under appropriate conditions as exemplified herein, a progenitor cell (as defined above) can be induced to differentiate into, e.g., an osteogenic precursor cell, which itself is capable of developing into one or more types of osteogenic cell; e.g., osteoblasts, osteocytes, etc.

The term “clonal” refers to a population of cells obtained by the expansion of a single cell into a population of cells all derived from that original single cell and not containing other cells.

For the purposes of the present disclosure, the terms “clonal progenitor cell”, “embryonic clonal progenitor cell”, “clonal progenitor cell line” and “embryonic clonal progenitor cell line” each refer to progenitor cell lines that are derived clonally, i.e., derived by the expansion of a single cell into a population of cells all derived from that original single cell and not containing other cells.

For the purposes of the present disclosure, the terms “osteoinductive” and “osteoinduction” refer to the process of inducing new bone formation de novo in an environment in which bone does not already exist. An example of an osteoinductive process is the formation of ectopic bone, in recipient tissue, following subcutaneous or intramuscular implantation of BMP-2.

For the purposes of the present disclosure, the terms “osteopromotive” and “osteopromotion” refer to the process of stimulating new bone growth from existing bone. For example, the action of osteoblasts can be considered to be osteopromotive.

The term “osteogenic” is intended to include both osteoinductive and osteopromotive processes.

A “cell-derived preparation” is a composition that is obtained from living cells and includes molecules from the cells, optionally also including residual live cells. Exemplary cell-derived preparations include lysates, extracts, lyophilisates, exosome preparations and conditioned medium. In some embodiments, a cell-derived preparation is obtained by treating the living cells in a way that breaks open or permeabilizes them (or otherwise causes them to release their contents) such that cellular contents are released, and no or very few living cells remain. Cell-derived preparations can be further fractionated to provide pure bioactive substances or mixtures thereof.

With respect to the production of cell-derived preparations (e.g., extracts, lysates, conditioned medium, exosomes, lyophilisates), the terms “physiological ratio” and “physiological proportions” refer to a mixture in which the various molecules produced by the cell (e.g., proteins, e.g., growth factors and cytokines) are present at the same relative levels as they are in the cell from which the cell-derived preparation was obtained. These terms are to be distinguished from “physiological concentration.” For example, due to dilution, the concentration of different molecules in an extract may be lower that their normal physiological concentrations, but they can still be present, with respect to one another, at normal physiological proportions. Similarly, concentration of a cell-derived preparation can lead to a solution containing supra-physiological concentrations of molecules that are present in normal physiological proportions with respect to one another.

For the purposes of the present disclosure, a “biological carrier” refers to any transplantable material to which cells, cell-derived preparations and bioactive substances can be adsorbed or applied to prior to transplantation. Exemplary biological carriers include collagen, hyaluronan, fibrin, elastin, hydrogels, gelatin, naturally-occurring extracellular matrix (ECM) (e.g., MatriGel®, amnion, demineralized bone matrix), synthetic ECM (e.g., recombinantly-produced collagen) and synthetic carriers such as, for example, polyglycolic acid (PGA), polylactic acid (PLA), polycaprolactone (PCL) and combinations thereof. Various ceramics such as, for example, hydroxyapatite and tricalcium phosphate, and collagen/ceramic composites, can also be used as biological carriers.

“Mesenchymal stem cells” or “mesenchymal stromal cells (MSCs)” or “marrow adherent stem cells” or “marrow adherent stromal cells (MASCs)” or “bone marrow stromal cells (BMSCs)” are multipotent cells that can be obtained, inter alia, from bone marrow and umbilical cord blood. MSCs normally differentiate into bone, cartilage and adipose tissue; and they can be separated from hematopoietic stem cells, in bone marrow aspirates, by their ability to attach to plastic substrates. MSCs express the surface markers CD73, CD90 and CD105; and do not express CD34, CD45, CD11b, CD14, CD79a, CD19 or HLA-DR. See Dominici et al. (2006), Cytotherapy 8(4): 315-317; Boxall and Jones, (2012) Stem Cells Int., 975871. MSCs further express the surface marker CD74, which is not expressed by the progenitor cells of the instant invention. See Barilleax et al. (2010), In Vitro Cell Dev Biol Anim. 46(6): 566-572; Sternberg et al., (2013) Regen. Med. 8(2): 125-144.

The present disclosure provides, inter alia, compositions comprising cell-derived preparations from osteogenic precursor cells (and/or chondrogenic precursor cells) and a biological carrier. Such compositions can be used to stimulate bone formation (and/or cartilage formation) in a human or animal subject by transplanting the composition to a site in the subject at which bone formation is required. Methods of making and using the compositions are also provided.

Osteogenic and chondrogenic precursor cells may be derived, for example, from the human embryonic progenitor (hEP) cell lines described infra.

Progenitor Cell Lines

The derivation and characterization of SK11, SM30, MEL2, 4D20.8 (sometimes referred to as X4D20.8), 7PEND24 (sometimes referred to as X7PEND24), 7SMOO32 (sometimes referred to as XSMOO32) and E15 human embryonic progenitor (hEP) cell lines has been described, e.g., in West et al., 2008 Regenerative Medicine 3(3), pp. 287-308, US Patent Application Publication No. 2010/0184033, Sternberg et al., (2013) Regen. Med. 8(2):125-144 and US Patent Application No. 2014/0234964, all of which are incorporated by reference herein in their entirety.

SK11

SK11 cells are positive for the markers: BEX1, COL21A1, FST, ICAM5, IL1R1, TMEM199, PTPRN, SERPINA3, SFRP2 and ZIC1 and are negative for the markers: ACTC, AGC1, ALDH1A1, AQP1, ATP8B4, C6, C20orf103, CCDC3, CDH3, CLDN11, CNTNAP2, DIO2, DKK2, EMID1, GABRB1, GSC, HOXA5, HSPA6, IF127, INA, KRT14, KRT34, IGFL3, LOC92196, MEOX1, MEOX2, MMP1, MX1, MYH3, MYH11, IL32, NLGN4X, NPPB, OLR1, PAX2, PAX9, PDE1A, PENK, PROM1, PTN, RARRES1, RASD1, RELN, RGS1, SMOC1, SMOC2, STMN2, TAC1, TFPI2, RSPO3, TNFSF7, TNNT2, TRH and TUBB4. SK11 cells are negative for the expression of MSC marker CD74.

Under appropriate conditions (e.g., grown in culture in the presence of BMP-2 or TGF-β3 or BMP-4, or combinations of these factors), SK11 cells are capable of differentiating into osteogenic precursor cells that express one or more markers chosen from bone sialoprotein II (TBSP), osteopontin (SPP1) and alkaline phosphatase, tissue-nonspecific isozyme (ALPL).

SM30

SM30 cells are positive for the markers: COL15A1, CRYAB, DYSF, FST, GDF5, HTRA3, TMEM119, MMP1, MSX1, MSX2, MYL4, POSTN, SERPINA3, SRCRB4D and ZIC2 and are negative for the markers: ACTC, AGC1, AKRIC1, ALDH1A1, ANXA8, APCDD1, AQP1, ATP8B4, CFB, C3, C6, C7, C20orf103, CD24, CDH3, CLDN11, CNTNAP2, COMP, DIO2, METTL7A, DKK2, DLK1, DPT, FGFR3, TMEM100, FMO1, FMO3, FOXF2, GABRB1, GJB2, GSC, HOXA5, HSD11B2, HSPA6, ID4, IF127, IL1R1, KCNMB1, KIAA0644, KRT14, KRT17, KRT34, IGFL3, LOC92196, MEOX1, MEOX2, MGP, MYBPH, MYH3, MYH11, NLGN4X, NPPB, OGN, OLR1, OSR2, PAX2, PAX9, PDE1A, PENK, PRG4, PROM1, PRRX1, PTN, RARRES1, RASD1, RELN, RGS1, SLITRK6, SMOC1, SMOC2, SNAP25, STMN2, TAC1, RSPO3, TNFSF7, TNNT2, TRH, TUBB4, UGT2B7 and WISP2. SM30 cells are negative for the expression of MSC marker CD74.

Under appropriate conditions (e.g., grown in culture in the presence of BMP-2 or TGF-β3 or BMP-4, or combinations of these factors), SM30 cells are capable of differentiating into osteogenic precursor cells that express one or more markers chosen from bone sialoprotein II (TBSP), osteopontin (SPP1) and alkaline phosphatase, tissue-nonspecific isozyme (ALPL).

MEL2

The cell line MEL2 is positive for the markers: AKR1C1, AQP1, COL21A1, CRYAB, CXADR, DIO2, METTL7A, DKK2, DLK1, DLX5, HAND2, HSD17B2, HSPB3, MGP, MMP1, MSX2, PENK, PRRX1, PRRX2, S100A4, SERPINA3, SFRP2, SNAP25, SOX11, TFPI2 and THY1 and is negative for the markers: ACTC, ALDH1A1, AREG, CFB, C3, C20orf103, CD24, CDH3, CDH6, CNTNAP2, COL15A1, COMP, COP1, CRLF1, FGFR3, FMO1, FMO3, FOXF2, FST, GABRB1, GAP43, GDF5, GDF10, GJB2, GSC, HOXA5, HSD11B2, HSPA6, ICAM5, KCNMB1, KRT14, KRT17, KRT19, KRT34, MASP1, MEOX1, MEOX2, MYBPH, MYH3, MYH11, TAGLN3, NPAS1, NPPB, OLR1, PAX2, PDE1A, PITX2, PRG4, PTN, PTPRN, RASD1, RELN, RGS1, SMOC1, STMN2, TACT, TNFSF7, TRH, TUBB4, WISP2, ZIC1 and ZIC2. MEL2 cells are negative for the expression of MSC marker CD74.

Under appropriate conditions (e.g., grown in culture in the presence of BMP-2 or TGF-β3 or BMP-4, or combinations of these factors), MEL2 cells are capable of differentiating into osteogenic precursor cells that express one or more markers chosen from bone sialoprotein II (TBSP), osteopontin (SPP1) and alkaline phosphatase, tissue-nonspecific isozyme (ALPL). 4D20.8

The cell line 4D20.8 is positive for the markers: BEX1, CDH6, CNTNAP2, COL21A1, CRIP1, CRYAB, DIO2, DKK2, GAP43, ID4, LAMC2, MMP1, MSX2, S100A4, SOX11 and THY1 and is negative for the markers: AGC1, ALDH1A1, AREG, ATP8B4, CFB, C3, C7, C20orf103, CDH3, CLDN11, COP1, CRLF1, DLK1, DPT, FMO1, FMO3, GDF10, GJB2, GSC, HOXA5, HSD11B2, HSD17B2, HSPA6, HSPB3, ICAM5, IF127, IGF2, KRT14, KRT17, KRT34, MASP1, MEOX2, MSX1, MX1, MYBPH, MYH3, MYH11, TAGLN3, NPAS1, NPPB, OGN, OLR1, PAX2, PDE1A, PRG4, PROM1, PTN, PTPRN, RARRES1, RGS1, SNAP25, STMN2, TAC1, TNNT2, TRH, TUBB4, WISP2, ZIC1 and ZIC2. 4D20.8 cells are negative for the expression of MSC marker CD74.

Under appropriate conditions (e.g., grown in culture in the presence of TGF-β3, or TGF-β3 plus BMP4 or TGF-β3 plus GDF5), 4D20.8 cells are capable of differentiating into chondrogenic precursor cells that express COL2A1 or ACAN.

7PEND24

The cell line 7PEND24 is positive for the markers: AQP1, BEX1, CDH3, DIO2, DLK1, FOXF1, FST, GABRB1, IGF2, IGFBP5, IL1R1, KIAA0644, MSX1, PODN, PRRX2, SERPINA3, SOX11, SRCRB4D and TFPI2 and negative for the markers: ACTC, AGC1, AKR1C1, ALDH1A1, ANXA8, APCDD1, AREG, CFB, C3, C6, C7, PRSS35, CCDC3, CD24, CLDN11, COMP, COP1, CXADR, DKK2, EMID1, FGFR3, FMO1, FMO3, GAP43, GDF10, GSC, HOXA5, HSD11B2, HSPA6, HTRA3, ICAM5, ID4, IFI27, IFIT3, INA, KCNMB1, KRT14, KRT17, KRT34, IGFL3, LOC92196, MFAP5, MASP1, MEOX1, MEOX2, MMP1, MX1, MYBPH, MYH3, MYH11, MYL4, IL32, NLGN4X, NPPB, OGN, OSR2, PAX2, PAX9, PENK, PITX2, PRELP, PRG4, PRRX1, RARRES1, RELN, RGMA, SFRP2, SMOC1, SMOC2, SODS, SYT12, TAC1, TNFSF7, TRH, TSLP, TUBB4, UGT2B7, WISP2, ZD52F10, ZIC1 and ZIC2. 7PEND24 cells are negative for the expression of MSC marker CD74.

Under appropriate conditions (e.g., grown in culture in the presence of TGF-β3, or TGF-β3 plus BMP4 or TGF-β3 plus GDF5), 7PEND24 cells are capable of differentiating into chondrogenic precursor cells that express COL2A1 or ACAN.

### 7SMOO32

The cell line 7SMOO32 is positive for the markers: ACTC, BEX1, CDH6, COL21A1, CRIP1, CRLF1, DIO2, DLK1, EGR2, FGFR3, FOXF1, FOXF2, FST, GABRB1, IGFBP5, KIAA0644, KRT19, LAMC2, TMEM119, MGP, MMP1,

MSX1, MSX2, PODN, POSTN, PRG4, PRRX2, PTN, RGMA, S100A4, SERPINA3, SOX11 and SRCRB4D and is negative for the markers: AGC1, AKR1C1, ALDH1A1, ANXA8, APCDD1, AREG, ATP8B4, BMP4, C3, C6, C7, PRSS35, C20orf103, CCDC3, CD24, CLDN11, CNTNAP2, COL15A1, COP1, CXADR, METTL7A, DKK2, DPT, EMID1, TMEM100, FMO1, FMO3, GDF5, GDF10, GJB2, GSC, HOXA5, HSD11B2, HSD17B2, HSPA6, HSPB3, HTRA3, ICAM5, ID4, IFI27, IL1R1, INA, KCNMB1, KRT14, KRT17, KRT34, IGFL3, LOC92196, MFAP5, MASP1, MEOX1, MEOX2, MYBPH, MYH3, MYH11, MYL4, IL32, NLGN4X, NPPB, OGN, OLR1, OSR2, PAX2, PAX9, PDE1A, PITX2, PRELP, PROM1, PTPRN, RASD1, RGS1, SFRP2, SMOC1, SMOC2, SOD3, STMN2, SYT12, TAC1, RSPO3, TNFSF7, TNNT2, TRH, TSLP, TUBB4, UGT2B7, WISP2, ZD52F10, ZIC1 and ZIC2. 7SMOO32 cells are negative for the expression of MSC marker CD74.

Under appropriate conditions (e.g., grown in culture in the presence of TGF-β3, or TGF-β3 plus BMP4 or TGF-β3 plus GDF5), 7SMOO32 cells are capable of differentiating into chondrogenic precursor cells that express COL2A1 or ACAN.

E15

The cell line E15 is positive for the markers: ACTC, BEX1, PRSS35, CRIP1, CRYAB, GAP43, GDF5, HTRA3, KRT19, MGP, MMP1, POSTN, PRRX1, S100A4, SOX11, SRCRB4D and THY1 and is negative for the markers: AGC1, AKR1C1, ALDH1A1, ANXA8, APCDD1, AQP1, AREG, ATP8B4, CFB, C3, C6, C7, C20orf103, CDH3, CNTNAP2, COP1, CXADR, METTL7A, DLK1, DPT, EGR2, EMID1, TMEM100, FMO1, FMO3, FOXF1, FOXF2, GABRB1, GDF10, GJB2, GSC, HOXA5, HSD11B2, HSD17B2, HSPA6, HSPB3, IFI27, IFIT3, IGF2, INA, KRT14, TMEM119, IGFL3, LOC92196, MFAPS, MASP1, MEOX1, MEOX2, MSX1, MX1, MYBPH, MYH3, MYL4, NLGN4X, TAGLN3, NPAS1, NPPB, OGN, OLR1, PAX2, PAX9, PDE1A, PENK, PITX2, PRG4, PROM1, PTPRN, RARRES1, RASD1, RELN, RGS1, SLITRK6, SMOC1, SMOC2, SNAP25, STMN2, TAC1, TFPI2, RSPO3, TNFSF7, TNNT2, TRH, TSLP, TUBB4, UGT2B7, WISP2, ZD52F10 and ZIC1. E15 cells are negative for the expression of MSC marker CD74.

Under appropriate conditions (e.g., grown in culture in the presence of TGF-β3, or TGF-β3 plus BMP4 or TGF-β3 plus GDF5), E15 cells are capable of differentiating into chondrogenic precursor cells that express COL2A1 or ACAN.

Osteogenic Precursor Cells

Osteogenic precursor cells (OPCs) are obtained, for example, by in vitro differentiation of progenitor cells such as, for example, SM30, MEL2 and SK11 cell lines. For example, culture of SM30 or MEL2 cells in the presence of one or more polypeptides from the TGF-beta superfamily induces differentiation of the progenitor cells into osteogenic precursor cells. Exemplary TGF-beta superfamily members include, but are not limited to, BMP-2, BMP-4, BMP-7 and TGF-β3. Exemplary culture conditions that can be used to convert progenitor cells to osteogenic precursor cells are described in the US Patent Application No. 2014/0234964.

In certain embodiments, clonal cultures of OPCs and cell-derived preparations from clonal cultures of OPCs are used in the manufacture of the compositions described herein. In certain embodiments, the cell-derived preparations described herein are not obtained from embryoid bodies.

Chondrogenic Precursor Cells

In certain embodiments, the compositions disclosed herein can contain cell-derived preparations obtained from chondrogenic precursor cells, either alone or in addition to cell-derived preparations from osteogenic precursor cells; thus providing a composite graft. Exemplary chondrogenic precursor cells are described in U.S. Patent Application Publication No. 2010/0184033, International Patent Application Publication No. WO 2013/010045 and U.S. Pat. No. 8,695,386, all of which are herein incorporated by reference in their entireties for the purposes of disclosing chondrogenic precursor cells and their properties. Bioactive factors obtained from chondrogenic cells can also be used as an alternative to, or in addition to, cell-derived preparations from chondrogenic cells in a composite graft.

Composite Grafts

A composite graft, containing cell-derived preparations from both osteogenic precursor cells and chondrogenic precursor cells, or bioactive factors derived therefrom, can be used for the treatment of various musculoskeletal conditions. These include, but are not limited to, osteochondritis dessicans (OCD) and other deep osteochondral joint defects (e.g., those resulting from a pathological condition, an injury or a surgically created defect from an osteochondral graft harvesting procedure).

In certain embodiments, composite grafts are made with two distinct layers: a bone-forming layer to anchor into host bone tissue, and a cartilage layer made of cartilage inducing bioactive to provide a friction-free joint motion surface. Such a composite graft can be press-fitted into deep osteochondral joint defects. Graft material is prepared by sequential loading of either osteogenic precursor cells (or a cell-derived preparation from osteogenic precursor cells) in a first layer or compartment of a biological carrier and chondrogenic precursor cells (or a cell-derived preparation from chondrogenic precursor cells) in a second layer of the biological carrier.

Composite grafts can be made from any combination of osteogenic and/or chondrogenic precursor cells, cell-derived preparations from osteogenic and/or chondrogenic precursor cells, or combinations of cells and cell-derived preparations.

Such composite grafts are useful, for example, in the treatment of osteochondral defects of joints such as the knee or the hip. The osteogenic portion of the graft would provide structural support and integrate with subchondral bone, while the chondrogenic portion would restore a cartilage defect in a joint, providing smooth friction-free motion. The composite graft could be machined or shaped in various cylindrical shape diameters to allow arthroscopic placement using standard osteochondral graft surgical tools. Alternatively, the larger flexible surface area graft could engineered to cover large defect area and adapt to the natural contour of the joint surface.

Biological Carriers

Transplantable biological carriers, to which cells and bioactive substances can be adsorbed prior to transplantation, are known in the art. See, for example, U.S. Patent Application Publication No. 2004/0062753, incorporated by reference. Exemplary biological carriers, for use in the manufacture of the disclosed compositions, include collagen (e.g., collagen sponges), hyaluronan, fibrin, elastin, hydrogels (see, e.g., Ahmed (2015) “Hydrogel: Preparation, Characterization and Applications: A Review,” J. Advanced Res. 6(2):105-121), gelatin, naturally-occurring extracellular matrix (ECM) (e.g., MatriGel®, amnion, demineralized bone matrix), synthetic ECM (e.g., recombinantly-produced collagen) and synthetic carriers such as, for example, polyglycolic acid (PGA), polylactic acid (PLA), polycaprolactone (PCL) and combinations thereof. Various ceramics such as, for example, hydroxyapatite and tricalcium phosphate, and collagen/ceramic composites, can also be used as biological carriers.

In certain embodiments, synthetic matrices or biological resorbable immobilization vehicles (sometimes referred to as “scaffolds”) are impregnated with progenitor cells, osteogenic precursor cells, and/or chondrogenic precursor cells as disclosed herein. A variety of carrier matrices have been used to date and include: three-dimensional collagen gels (U.S. Pat. No. 4,846,835; Nishimoto (1990) Med. J. Kinki University 15:75-86; Nixon et al. (1993) Am. J. Vet. Res. 54:349-356; Wakitani et al. (1989) J. Bone Joint Surg. 718:74-80; Yasui (1989) J. Jpn. Ortho. Assoc. 63:529-538); reconstituted fibrin-thrombin gels (U.S. Pat. Nos. 4,642,120; 5,053,050 and 4,904,259); synthetic polymer matrices containing polyanhydride, polyorthoester, polyglycolic acid and copolymers thereof (U.S. Pat. No. 5,041,138); hyaluronic acid-based polymers (Robinson et al. (1990) Calcif. Tissue Int. 46:246-253); and hyaluronan and collagen-based polymers such as HyStem®-C(BioTime), e.g., as described in U.S. Pat. Nos. 7,981,871 and 7,928,069, the disclosures of which are herein incorporated by reference. HyStem®-C may be employed in numerous applications in which the prevention of undesired inflammation or fibrosis is desired, such as in the repair of traumatic orthopedic injuries such as tears to rotator cuff tendons, carpal tunnel syndrome, and trauma to tendons generally.

Osteogenic and/or chondrogenic precursor cells, as disclosed herein, can be employed in tissue reconstruction as described in Methods of Tissue Engineering (2002), edited by Anthony Atala and Robert P. Lanza and published by Academic Press (London), incorporated by reference herein for its description of tissue reconstruction (see, e.g., pages 1027 to 1039). For example, cells can be placed into a molded structure (e.g., by injection molding) and transplanted into a subject. Over time, tissue produced by the cells will replace the molded structure, thereby producing a formed structure (i.e., in the shape of the initial molded structure). Exemplary mold materials for the molded structure include hydrogels (e.g., alginate, agarose, polaxomers (Pluronics)) and natural materials (e.g., type I collagen, type II collagen, and fibrin).

In certain embodiments, the biological carrier is demineralized bone matrix (DBM). In other embodiments, the biological carrier is not demineralized bone matrix.

Cell-Derived Preparations

Graft-forming units, as disclosed herein, comprise a biological carrier, combined with a cell-derived preparation from an osteogenic precursor cell and/or a cell-derived preparation from a chondrogenic precursor cell. The cell-derived preparation can be, for example, a lyophilisate, a lysate, an extract, an exosome preparation, and/or a preparation of conditioned medium. Such cell-derived preparations will contain mixtures of bioactive substances in their normal physiological proportions with respect to one another. Since processes such as ossification often depend upon a plurality of factors, each present at optimal concentration, compositions such as those described herein, containing physiological proportions of bioactive factors, will be maximally effective.

Cell-derived preparation can, in certain circumstances, comprise a small number of residual live cells. Methods for estimating the live cell content of a cell-derived preparation include, for example, Trypan Blue staining and LDH release assays. In certain embodiments, a cell-derived preparation contains less than 5% viable cells, compared to the number of cells from which the cell-derived preparation was obtained. In additional embodiments, a cell-derived preparation contains less than 4%, less than 3%, less than 2%, less than 1%, less than 0.5%, less than 0.25%, less than 0.1%, or less than 0.05% viable cells, or contains no viable cells at all.

Lysates

Methods for preparation of cell lysates are well-known in the art. Physical methods include, for example, mechanical disruption of the cell membrane, such as using a blender or homogenizer, sonication, freeze-thawing and manual grinding. Chemical methods include treating cells with detergents such as, for example, SDS, Triton X-100, Triton N-101, Triton X-114, Triton X-405, Triton X-70S, Triton DF-16, monolaurate (Tween 20), monopalmitate (Tween 40), mono-oleate (Tween 30 80), polyoxyethylene-23-lauryl ether (Brij 35), polyoxyethylene ether W-1 (Polyox), sodium cholate, deoxycholates, CHAPS, saponin, n-Decyl ˜-D-glucopuranoside, n-heptyl ˜-D glucopyranoside, n-Octyl a-D-glucopyranoside and Nonidet P-40.

In certain embodiments, physical methods for lysis are used because they do not remove or inactivate growth factors. By contrast, detergents can form complexes with growth factors that can be difficult to reverse. Lower concentrations of detergents can be used to minimize this problem, for example, as are found in RIPA or CellLytic buffers (Sigma, St. Louis, Mo.). A combination method that utilizes both physical lysis with a small amount of detergent can also be used.

In an exemplary method of obtaining a freeze-thaw lysate, cells are cultured, and optionally differentiated, to obtain the desired number of precursor cells. Precursor cells are removed from the culture vessel with Trypsin, rinsed with saline, and subjected to centrifugation to remove Trypsin. The cell pellet is washed to remove saline and resuspended in a volume of water sufficient to cover the cell pellet. The cells are held at a temperature of −20° C. or less (e.g., for 30 minutes), then thawed (e.g., at 37° C. or room temperature). This freeze/thaw cycle can be repeated one or more times (e.g., three times), as necessary. Following the desired number of freeze/thaw cycles, the lysate is subjected to centrifugation at 13,000 rpm. The pellet contains cell membrane debris and the supernatant contains cellular proteins. In certain embodiments, the freeze/thaw cycles are conducted in the presence of small amounts of detergent (e.g., 0.1% Triton X-100) to help release proteins (e.g., growth factors) from the membrane and into the supernatant.

An alternative method for obtaining a freeze/thaw lysate is to culture, and optionally differentiate, cells on a biological carrier (e.g., a scaffold) to obtain the desired number of precursor cells. The cell-containing scaffold is rinsed extensively with saline and centrifuged. Saline is removed and a volume of water sufficient to cover the cell-seeded scaffold is added. The cell-containing scaffolds are frozen at −20° C. (e.g., for 30 minutes) and thawed at 37° C. or room temperature. The freeze/thaw cycle can be repeated as necessary and, following a desired number of cycles, the preparations are optionally lyophilized. Using this method, cell membranes are retained on the scaffold, which might prove advantageous for the recovery of surface molecules (e.g., membrane proteins).

To obtain a lysate by sonication, cells are cultured and optionally differentiated to obtain the desired number of precursor cells, then removed from the tissue culture vessel (e.g., with Trypsin). The cells are centrifuged and washed (e.g., three times) with an excess volume of PBS or saline to remove culture medium and trypsin. The final cell pellet is resuspended (e.g., in PBS, water or saline) and placed on ice. Alternatively, cells are resuspended in buffer containing protease inhibitors, for example, 50 mM Tris-HCl pH 7.5, 10 μg/mL Antipain, 0.5 μM Pepstatin, 0.1 mM DTT, 0.1 mM PMSF. Sonication is conducted using, for example, a Soniprep (MSE, London, UK) or a Branson sonifier (Emerson Industrial, Danbury, Conn.) with 3 cycles of 15 seconds on, 5 seconds off at 20% power while samples are kept on ice. Alternatively, 3 bursts of 5 seconds on with 25 second intervals using 15 amplitude micron power can be used. Those skilled in the art recognize that sonication methods can be optimized by altering the pulse times, number of iterations and pulse intensity. Sonicated samples are subjected to centrifugation at 15,000rcf for 5 minutes and the supernatant is collected. The supernatant contains intracellular molecules (e.g., proteins) and the pellet contains cell membrane. A small amount of detergent (e.g., 0.1% Triton X-100) can be included to help release growth factors and other surface molecules from the membrane and into the supernatant.

For both freeze/thaw lysates and lysates obtained by sonication, the supernatant volume can be adjusted to obtain a desired protein concentration. Alternatively, standard methods for concentrating proteins can be used. For example, Centricon (EMD Millipore, Temecula, Calif.) is a centrifugation/filtration method used to reduce volume while retaining proteins. Protein precipitation using ammonium sulfate, trichloroacetic acid, acetone or ethanol are also routinely used to concentrate proteins.

In certain embodiments, a lysate-coated biological carrier is obtained by adding a saturating concentration of a lysate to a dry biological carrier and lyophilizing the lysate-coated biological carrier.

Lyophilisates

Methods for lyophilization (i.e., freeze-drying) are known in the art and comprise subjecting a sample to reduced pressure and temperature.

An exemplary method for obtaining a lyophilizate of an osteogenic precursor cell is to apply a suspension of osteogenic precursor cells to a biological carrier (or grow osteogenic precursor cells on a biological carrier) and lyophilize the cell-seeded carrier.

Extracts

In additional embodiments, lysates of osteogenic and/or chondrogenic precursor cells are further purified or fractionated to provide a cell extract. Methods for making extracts of mammalian cells are known in the art. The extract can then be applied to a biological carrier, and the extract-seeded carrier is optionally lyophilized.

As used herein, “extract” refers to a solution obtained from a cell culture, cell lysate, cell pellet, cell supernatant or cell fraction by the use of a solvent (e.g., water, detergent, buffer, organic solvent) and optionally separated by, e.g., centrifugation, filtration, column fractionation, ultrafiltration, phase partition or other method. Exemplary solvents that can be used in the preparation of cell extracts include, but are not limited to, urea, guanidinium chloride, guanidinium isothiocyanate, sodium perchlorate and lithium acetate.

Conditioned Medium

In additional embodiments, conditioned medium is prepared from cultures of osteogenic and/or chondrogenic precursor cells, and the conditioned medium is optionally further purified or fractionated. The conditioned medium, or fraction thereof, is applied to a biological carrier and the saturated carrier is optionally lyophilized.

Methods for obtaining conditioned medium from mammalian cell cultures are known in the art. In general, cells are cultured under conditions appropriate for proliferation or differentiation, as desired. Cells are then removed from the culture vessel, washed and re-plated in a small volume of culture medium, for example, DMEM+Glutamax (Gibco/Invitrogen, Carlsbad, Calif.). The cells are cultured (e.g. for 24-48 hours) and the medium is collected to provide conditioned medium.

Conditioned medium can be obtained at various stages of differentiation and/or various times of culture. For example, conditioned medium can be obtained from progenitor cells (e.g., SM30 cells, MEL2 cells), or conditioned medium can be obtained from precursor cells (e.g., osteogenic and/or chondrogenic precursor cells). Alternatively, conditioned medium can be obtained at one or more stages during the differentiation of a progenitor cell to a precursor cell. Alternatively, conditioned medium can be obtained from cells (e.g., progenitor cells or precursor cells) that have been cultured, under non-differentiating conditions, for various amounts of time.

Once harvested, conditioned medium can optionally be further processed by concentration or fractionation, using standard techniques known to those of skill in the art. Concentration is achieved, for example, by harvesting culture medium and submitting said medium to ultrafiltration.

Exosomes

Exosomes are membrane-bound vesicles ranging from 30 to 120 nm and secreted by a wide range of mammalian cell types. Keller et al., (2006) Immunol. Lett. 107 (2): 102; Camussi et al., (2010) Kidney International 78:838. Exosomes are found both in cells growing in vitro as well as in vivo. They can be isolated from tissue culture media as well as bodily fluids such as plasma, urine, milk and cerebrospinal fluid. George et al., (1982) Blood 60:834; Martinez et al., (2005) Am J. Physiol. Health. Cir. Physiol 288:H1004. Exosomes contain a variety of molecules synthesized by the cell, including nucleic acids such as mRNA and miRNA and proteins such as various growth and/or differentiation factors.

Exosomes originate from the endosomal membrane compartment. They are stored in intraluminal vesicles within multivesicular bodies of the late endosome. Multivesicular bodies are derived from the early endosome compartment and contain within them smaller vesicular bodies that include exosomes. Exosomes are released from the cell when multivesicular bodies fuse with the plasma membrane. Methods for isolating exosomes from cells are known in the art and have been described, e.g., in US Patent Application Publication No. 2012/0093885; Lamparski et al., (2002) J. Immunol. Methods 270(2):211-226; Lee et al., (2012) Circulation 126(22):2601-2611; Böing et al., (2013) J. Extracell Vesicles 3:23430 and Welton et al., (2015) J. Extracell Vesicles 4:27269. An exemplary method for preparing exosomes from osteogenic precursor cells is provided in Example 4 below.

Exosomes can be obtained at various stages of differentiation and/or various times of culture. For example, exosomes can be obtained from progenitor cells (e.g., SM30 cells, MEL2 cells), or exosomes can be obtained from precursor cells (e.g., osteogenic and/or chondrogenic precursor cells). Alternatively, exosomes can be obtained at one or more stages during the differentiation of a progenitor cell to a precursor cell. Alternatively, exosomes can be obtained from cells (e.g., progenitor cells or precursor cells) that have been cultured, under non-differentiating conditions, for various amounts of time.

In certain embodiments, a preparation of exosomes is applied to a biological carrier and the exosome-saturated carrier is optionally freeze-dried. Exosome suspensions can be applied, optionally aseptically, at various concentrations ranging from 10 million, 100 million, 1 billion, 10 billion, or 100 billion particles/cc (or any integral value therebetween) or more of sterilized matrix. Freeze-drying stabilizes the exosome-derived bioactive factors adsorbed by the matrix support such that they can be maintained indefinitely at room temperature.

Purified and Recombinant Factors:

Any of the aforementioned cell-derived preparations can be further fractionated, by methods well-known in the art (e.g., phase partition, centrifugation, size exclusion, chromatography, HPLC), and/or by methods that separate molecules according to molecular weight, charge density, or relative solubility in various solutions, to provide fractions containing one or more bioactive factors. Such fractions can be combined with a biological carrier to provide a graft-forming unit.

In addition, one or more recombinant proteins can be combined with a biological carrier to provide a graft-forming unit. For example, the family of bone morphogenetic proteins (BMPs) are known to stimulate bone formation. Accordingly, a biological carrier can be combined with one or more BMP family members (e.g., BMP-2, BMP-4, BMP-7, BMP-12, BMP-14/GDF-5) and used for stimulation of bone formation after transplantation.

Methods of Making

The compositions of the invention comprise combinations of (1) a cell-derived preparation of an osteogenic and/or chondrogenic precursor cell with (2) a biological carrier, and combinations of (1) one or more bioactive substances with (2) a biological carrier. The combinations can be assembled simply by application of cells, lysates, extracts, conditioned medium, exosomes or bioactive substances to the carrier, optionally followed by, e.g., lysis and/or lyophilization, or a carrier can be placed in culture with cells and recovered after a predetermined time. The cell-seeded carrier can then be prepared for storage (e.g., by lyophilization) or treated in a way that releases intracellular contents which remain adsorbed to the carrier. In the latter case, optionally membrane proteins are removed from the carrier prior to storage and use; since membrane proteins can contribute to inflammatory responses in the transplant recipient.

Uses

The methods and compositions disclosed herein can be used, inter alia, to supplement bone grafting spinal fusion procedures, or for trauma and orthopedic bone reconstruction. A graft-forming unit, as described herein, can be utilized by itself to heal defects or in combination, e.g., to augment an autologous bone graft. For example, autologous bone grafts derived locally from bone shavings are lower quality than autologous bone derived from the iliac crest. Thus, the graft-forming units disclosed herein provide an off-the-shelf bone grafting product that would supplant the use of autologous bone grafts for orthopedic bone repair procedures, thereby avoiding the painful and risky process of harvesting autologous bone. Alternatively, the disclosed compositions can be used in combination with autologous bone shavings to augment bone healing and fusion.

Additional indications include bone trauma, craniomaxillofacial reconstruction and bone repair of extremities (e.g., foot and/or ankle arthrodeses).

The use of cell-derived graft-forming units has a number of advantages, compared to therapeutic compositions comprising live cells. For example, cell-derived compositions can be sterilized, permitting longer shelf life and/or the ability to be stored at room temperature. Additionally, the cGMP manufacturing, storage and transport logistics are simplified with cell-derived graft-forming units, and thus, the cost of goods is expected to be substantially reduced as well. The risk of tumor and/or teratoma formation (resulting from transplantation of viable cells) is also reduced with the use of cell-derived compositions.

Systems and Kits

In certain embodiments, cell-derived preparations and/or bioactive substances, optionally lyophilized or stabilized, are applied to a biological carrier at the point of care. Accordingly, the present disclosure provides systems and kits comprising (1) a cell-derived preparation from an osteogenic precursor cell and/or a cell-derived preparation from a chondrogenic precursor cell and (2) a biological carrier. The systems and kits may further include reagents and materials for the propagation and use of the cells for research and/or therapeutic applications as described herein.

Biological Deposits

Cell lines described in this application have been deposited with the American Type Culture Collection (“ATCC”; P.O. Box 1549, Manassas, Va. 20108, USA) under the Budapest Treaty. The cell line 4D20.8 (also known as ACTC84) was deposited at the ATCC at passage 11 on Jul. 23, 2009 and has ATCC Accession No. PTA-10231. The cell line SM30 (also known as ACTC256) was deposited at the ATCC on Jul. 23, 2009 at passage 12 and has ATCC Accession No. PTA-10232. The cell line 7SMOO32 (also known as ACTC278) was deposited at the ATCC at passage 12 on Jul. 23, 2009 and has ATCC Accession No. PTA-10233. The cell line E15 (also known as ACTC98) was deposited at the ATCC at passage number 20 on Sep. 15, 2009 and has ATCC Accession No. PTA-10341. The cell line MEL2 (also known as ACTC268) was deposited at the ATCC at passage number 22 on Jul. 1, 2010 and has ATCC Accession No. PTA-11150. The cell line SK11 (also known as ACTC250) was deposited at the ATCC at passage number 13 on Jul. 1, 2010 and has ATCC Accession No. PTA-11152. The cell line 7PEND24 (also known as ACTC283) was deposited at the ATCC at passage number 11 on Jul. 1, 2010 and has ATCC Accession No. PTA-11149.

### EXAMPLES

The following examples are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1: Differentiation of Human Embryonic Progenitor (hEP) Cells into Osteogenic and Chondrogenic Precursors

The hEP cell lines SM30, 4D20.8, and MEL2 can be converted to osteogenic precursors in vitro, as described in the following exemplary methods.

Differentiation to Osteogenic Precursors in Gels Containing Gelatin and Vitronectin

Tissue culture plates were exposed to 12 μg/mL of Type I collagen (gelatin) and 12 μg/mL of vitronectin for 24 hours. The gelatin/vitronectin solution was then aspirated and cells (SM30 or MEL2) were added at confluent density. Osteogenic medium comprising: DMEM (low glucose) with L-Glutamine, 10% fetal bovine serum, 0.1 μM dexamethasone, 0.2 mM ascorbic acid 2-phosphate, 10 mM glycerol-2-phosphate, and 100 nM BMP7 was added and cells were further cultured for 15-21 days.

The degree of osteogenesis was scored by relative staining with Alizarin red S performed as follows: Alizarin red S (Sigma) (40 mM) is prepared in distilled water and the pH is adjusted to 4.1 using 10% (v/v) ammonium hydroxide. Monolayers in 6-well plates (10 cm²/well) were washed with PBS and fixed in 10% (v/v) formaldehyde (Sigma-Aldrich) at room temperature for 15 min. The monolayers were then washed twice with excess distilled water prior to addition of 1 mL of 40 mM Alizarin red S (pH 4.1) per well. The plates were incubated at room temperature for 20 min with gentle shaking. After aspiration of the unincorporated dye, the wells were washed four times with 4 mL water while shaking for 5 min. The plates were then left at an angle for 2 min to facilitate removal of excess water, reaspirated, and then stored at −20° C. prior to dye extraction. Stained monolayers were visualized by phase-contrast microscopy using an inverted microscope (Nikon). For quantification of staining, 800 μL 10% (v/v) acetic acid was added to each well, and the plate was incubated at room temperature for 30 min with shaking. The monolayer (loosely attached to the plate) was scraped from the plate with a cell scraper (Fisher Lifesciences) and transferred with 10% (v/v) acetic acid to a 1.5-mL microcentrifuge tube with a wide-mouth pipette. After vortexing for 30 sec, the slurry was overlaid with 500 μL mineral oil (Sigma-Aldrich), heated to exactly 85° C. for 10 min, and transferred to ice for 5 min. Tubes were not opened until fully cooled. The slurry was then centrifuged at 20,000 g for 15 min; and 500 μL of the supernatant was removed to a new 1.5 mL microcentrifuge tube. 200 μL of 10% (v/v) ammonium hydroxide was added to neutralize the acid. The pH was measured at this point to ensure that it was between 4.1 and 4.5. Aliquots (150 μL) of the supernatant were assayed, in triplicate, by spectrophotometry at 405 nm in 96-well format using opaque-walled, transparent-bottomed plates (Fisher Lifesciences) as described (Gregory, C A et al., (2004) Analytical Biochemistry 329:77-84.

Differentiation to Osteogenic Precursors in Gels Containing Crosslinked Hyaluronic Acid and Gelatin

The cell lines disclosed herein can also be differentiated within hydrogels, including crosslinked gels containing hyaluronic acid and gelatin, with or without added growth and/or differentiation factors (see, for example, U.S. Patent Application Publication No. 2014/0234964). In this method, cells are trypsinized, then suspended at a concentration of 1-30×10⁶ cells/mL in HyStem-CSS (Glycosan Hydrogel Kit GS319) according to manufacturer's directions.

HyStem-CSS is prepared as follows. HyStem (thiol-modified hyaluranan) is dissolved in 1 mL degassed deionized water (taking about 20 minutes). Gelin-S(thiol modified gelatin) is dissolved in 1 mL degassed deionized water and PEGSSDA (disulfide-containing PEG diacrylate) is dissolved in 0.5 mL degassed deionized water (designated herein as “PEGSSDA solution”). The HyStem (1 mL) is mixed with the Gelin-S (1 mL), without creating air bubbles, immediately before use (designated herein as “HyStem: Gelin-S mix”).

For differentiation in HyStem hydrogel containing retinoic acid (RA) and epidermal growth factor (EGF), 1.7×10⁷ cells are pelleted and resuspended in 1.4 mL Hystem: Gelin-S mix. Then 0.35 mL of PEGSSDA solution is added, pipetted up and down, without creating air bubbles, and 100 ul aliquots are quickly placed onto multiple 24 well inserts (Corning Cat #3413). After gelation occurs, in approximately 20 minutes, encapsulated cells are fed 2 mL growth medium with all-trans-RA (1 μM) (Sigma, Cat #2625) or 2 mL growth medium with EGF (100 ng/mL) (R&D systems Cat#236-EG). Cells are fed three times weekly for approximately 28 days. At this time or later, cells can lysed and RNA can be harvested (e.g., using RNeasy micro kits (Qiagen Cat #74004)) for qPCR or microarray analysis, if desired.

Differentiation in Hydrogels Containing Crosslinked Hyaluronic Acid and Gelatin to Induce Chondrogenesis

Cells are suspended at a density of 2×10⁷ cells/mL in 1.4 mL Hystem:Gelin-S mix. Then, 0.35 mL of PEGSSDA solution is added, pipetted up and down without creating air bubbles, and 100 μl aliquots are quickly placed onto multiple 24 well inserts (Corning Cat #3413). After gelation has occurred, in approximately 20 minutes, encapsulated cells are fed 2 mL Complete Chondrogenic Medium which consists of Lonza Incomplete Medium plus TGF-beta3 (Lonza, PT-4124). Incomplete Chondrogenic Medium consists of hMSC Chondro BulletKit (PT-3925) to which is added supplements (Lonza, Basel, Switzerland, Poietics Single-Quots, Cat. # PT-4121). Supplements added to prepare Incomplete Chondrogenic Medium are: Dexamethasone (PT-4130G), Ascorbate (PT-4131G), ITS+supplements (4113G), Pyruvate (4114G), Proline (4115G), Gentamicin (4505G), and Glutamine (PT-4140G). Sterile lyophilized TGF-beta3 is reconstituted with the addition of sterile 4 mM HCl containing 1 mg/mL bovine serum albumin (BSA) to a concentration of 20 μg/mL and is stored in aliquots at −80° C. Complete Chondrogenic medium is prepared just before use by the addition of 1 μl of reconstituted TGF-beta3 for each 2 mL of Incomplete Chondrogenic medium (final TGF-beta3 concentration is 10 ng/mL). Cells are re-fed three times a week and cultured for a total of 14 days. Cells can then be lysed and RNA harvested using RNeasy micro kits (Qiagen Cat #74004), if desired.

### Example 2: Collagen-Containing Graft

To test the osteogenic potential of grafts containing osteogenic precursor cells, a nude rat osteoinduction model was used. Briefly, graft-forming units composed of lysates from cell lines SM30 or MEL2, previously isolated and characterized as described (West et al., Regen. Med. 3: 287-308 (2008)), combined with collagen sponge scaffolds, were implanted in an intramuscular pouch in the back of nude rats, and the extent of ectopic bone formation was assessed.

The cell lines were propagated independently as monolayer and expanded using conditions described previously (Sternberg et al., 2013 Regen. Med. 8(2): 125-144; U.S. Patent Application Publication No. 2014/0234964). After tissue culture expansion, the cells were dissociated with 0.083% trypsin-EDTA (Gibco Life Technologies, NY) (SM30) or Accutase (Gibco, NY) (MEL2) at 37° C. for 3-5 minutes, and resuspended in growth medium (PromoCell, Germany) at 0.5×10⁶ cells/100 μl. A dry collagen sponge (dimensions 1×1×0.5 cm, DANE Industrial Technologies Inc., NJ) was placed into each well of a 24-well ultralow cluster plate (Corning, Mass.) with the pore side of the collage sponge facing up. Approximately 100 μl of cells were seeded into each collagen sponge drop by drop, and allowed to settle at room temperature for 10-15 minutes. Then 1.5 mL of growth medium was added into each well, and cells were maintained overnight in a 37° C. incubator with 5% O₂ and 10% CO₂.

To induce osteogenic differentiation, cell-loaded collagen sponges were treated for 14 days with Induction Medium consisting of Dulbecco's Modified Eagle Medium (Corning, Mass.) supplemented with 1×ITS (BD Bioscience, CA), 2 mM Glutamax (Gibco), 100U/mL penicillin, 100m/mL Streptomycin (Gibco), 1 mM sodium pyruvate (Gibco), 100 nM dexamethasone(Sigma), 0.35 mM L-proline (Sigma), 0.17 mM 2-phospho-L-ascorbic acid (Sigma), 10 mM β-glycerophosphate(Sigma), 100 ng/mL BMP2 (Humanzyme, IL) and 10 ng/mL TGF-beta3 (R&D Systems, MN). At day 14, medium was aspirated and the cell-seeded sponges were washed once with PBS, then lyophilized in a FreeZone 2.5 (Labcono, Kansas City, Mo.) for 16-24 hours prior to implantation.

Negative control sponges (not containing cells) were treated in growth medium or Induction medium for 14 days, at which time the medium was removed, the sponges were washed once with PBS and lyophilized as described above for the cell-seeded sponges.

For positive controls, 50 μl of a solution containing recombinant human Bone Morphogenetic Protein-2 (rhBMP-2) (R&D labs), dissolved in sterile water to a concentration of 0.3 μg/μl, was added to a dry collagen sponge and allowed to absorb for 20 minutes prior to implantation.

The grafts were then implanted in a surgically created pouch in the dorsal muscle of immuno-compromised NIH-Foxn1^{rnu/rnu} rats (which do not raise an immune response against human antigens). Prior to implantation, 50 μl of water was added to each sponge (experimental and control). Four replicate implants per rats were used, two in the thoracic (chest) and two lumbar area of the back, as follows. Animals were anesthetized according to established UCSD IACUC-approved procedures, and prepared for surgery as described in UCSD IACUC guidelines. The incision sites were shaved and sanitized with betadine & alcohol. A posterior midline incision was made in the skin. Two separate paramedian incisions were made 3 mm from the midline in the lumbar fascia and thoracic paravertebral fascia, and two intramuscular pouches at each level were created through these incisions. The grafts were implanted into each intramuscular pouch. The subcutaneous tissue was sutured with 4.0 Vicryl and the skin was closed with staples. The animals were given antibiotics, recovered from anesthesia and returned to their cages. One day post-op the animals received Buprenex® (0.05 mg/kg IP) for analgesia. The rats were kept ad libitum in their cage afterwards.

Bone formation was assayed at four weeks and six weeks after surgery. MicroCT scans were performed and a qualitative scoring from − (no bone) to +++(extensive bone formation) was used to quantify outcome. Results are shown in Table 1.

TABLE 1
Bone formation after implantation of whole-cell grafts in rats

| | | 4 Weeks | | 6 Weeks | |
|---|---|---|---|---|---|
| MRI* | Condition | Chest | Lumbar | Chest | Lumbar |
| 6 | Negative | + and + | + and − | + and + | + and − |
| | control | | | | |
| 12 | SM30 cells | ++ and ++ | ++ and ++ | +++ and +++ | +++ and +++ |
| 13 | MEL2 cells | ++ and ++ | ++ and ++ | ++ and ++ | ++ and ++ |
| 15 | BMP2 | ++ and ++ | + and ++ | ++ and ++ | +++ and +++ |

*Animal code

Six weeks after surgery, all animals were euthanized using CO₂. The implants were then visually localized and excised, together with some surrounding soft tissue, using scalpels and forceps. Excised implants were fixed in 10% neutral buffered formalin, decalcified, paraffin-embedded, and longitudinally sectioned (4 μm). Serial sections were stained with hematoxylin and eosin (H&E) or Masson's trichrome. The histological images were digitally captured using the Leica SCN400 Slide Scanner at 40× magnification (Leica Microsystems, Milton Keynes, UK).

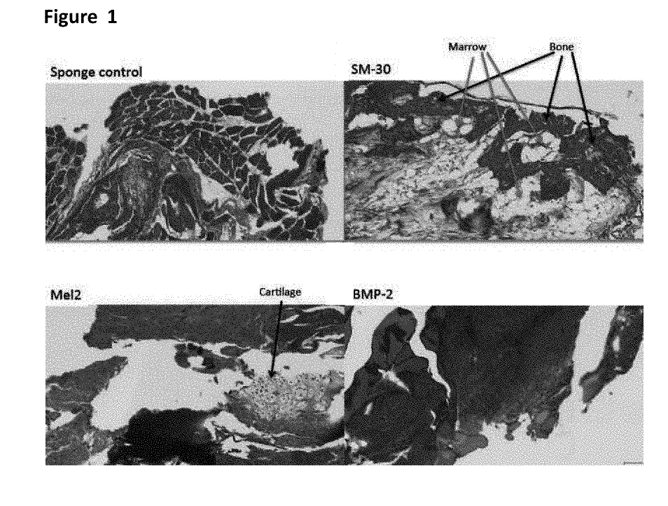

The results, shown in FIG. 1, demonstrate surprising bone formation resulting from transplantation of lyophilized collagen sponges on which SM30 and MEL2 cells were cultured in inductive medium for 14 days. The bone formation properties of cell-seeded sponges were superior compared to the collagen sponge control used under identical conditions. Bone formation induced by cell-seeded sponges was comparable to, or superior than, that obtained using a known osteoinductive protein, BMP-2, on a collagen sponge.

### Example 3: Hydrogel-Containing Graft

The cell line SM30 (passage 22) was differentiated in HyStem hydrogel which is a PEGDA crosslinked polymer of hyaluronic acid and gelatin according to manufacturer's instructions (Glycosan) for 14 days in the presence of 10 ng/mL of TGF-β3. SM30 cells were expanded in vitro for >21 doublings, synchronized in quiescence by growing to confluence and replacing the media with media supplemented with a 10-fold reduction in serum or other mitogens as described herein (CTRL), or differentiated in micromass conditions as described herein (MM), or differentiated in HyStem hydrogel which is a PEGDA crosslinked polymer of hyaluronic acid and gelatin according to manufacturer's instructions (Glycosan) for 14 days in the presence of either 10 ng/mL of TGF-β3, 25 ng/mL TGF-β3, 10 ng/mL BMP4, 30 ng/mL BMP6, 100 ng/mL BMP7, 100 ng/mL GDF5, or combinations of these growth factors. In brief, the hydrogel/cell formulation was prepared as follows: HyStem (Glycosan, Salt Lake, Utah, HyStem-CSS Cat #GS319) was reconstituted following manufacturer's instructions. Briefly, Hystem (thiol modified hyaluronan, 10 mg) was dissolved in 1 mL degassed deionized water (taking about 20 minutes) to prepare a 1% solution. Gelin-S(thiol modified gelatin, 10 mg) was dissolved in 1 mL degas sed deionized water to prepare a 1% solution, and PEGSSDA (disulfide-containing PEG diacrylate, 10 mg) was dissolved in 0.5 mL degassed deionized water to prepare a 2% solution. Then HyStem (1 mL, 1%) is mixed with Gelin-S (1 mL, 1%) without creating air bubbles, immediately before use. Pelleted cells were resuspended in recently prepared HyStem Gelin-S (1:1) mix described above. Upon the addition of crosslinker PEGSSDA (disulfide containing polyethylene glycol diacrylate), 100 μl of the cell suspension, at a final concentration of 20×10⁶ cells/mL, is aliquoted into multiple 24 well plate, 6.5 mm polycarbonate (0.4 μM pore size) transwell inserts (Corning 3413). Following gelation in about 20 minutes, chondrogenic medium is added to each well. Plates are then placed in humidified incubator at 37° C., ambient O₂, 10% CO₂, and cells are fed three times weekly. Under these conditions, SM30, in the presence of 50.0 ng/mL BMP2 and 10 ng/mL TGF-β3, and 10 mg/mL BMP4 and 10 ng/mL TGF-β3, expressed relatively high levels of bone sialoprotein II (IBSP, a molecular marker of bone-forming cells) and very high levels of COL2A1 and COL10A1, suggesting intermediate hypertrophic chondrocyte formation (i.e. endochondral ossification). Lesser, but nevertheless elevated levels of IBSP expression was also observed in the cell line MEL2 in pellet culture in 10 ng/mL TGF-β3.

### Example 4: Preparation of Exosomes

SM30, 4D20.8 or MEL2 cells are induced to differentiate into osteogenic precursor cells as described in Example 1. Exosomes are isolated from medium conditioned by the osteogenic precursor cells cultured in a humidified tissue culture incubator for 16 hours at 37° C. with 5% CO₂ and 1% O₂. Phosphate-buffered saline (PBS) is added to the cultures to a final concentration of 0.1 mL/cm² to produce conditioned medium. Alternatively basal EGM medium (Promocell, Heidelberg, Germany) without fetal calf serum or growth factors additives is substituted for PBS. The media is conditioned by the cells in a humidified tissue culture incubator for 16 hours at 37° C. at 5% CO₂ and 1% O₂. Phosphate-buffered saline (PBS) is added to the cultures to a final concentration of 0.1 mL/cm² to produce conditioned medium. Alternatively basal EGM medium (Promocell, Heidelberg, Germany) without fetal calf serum or growth factors additives is substituted for PBS.

The conditioned medium is collected and 0.5 volumes of Total Exosome Isolation Reagent (Life Technologies, Carlsbad, Calif.) is added and mixed well by vortexing until a homogenous solution is obtained. Alternatively a solution consisting of 15% polyethylene glycol/1.5 M NaCl is substituted for the Total Exosome Isolation Reagent. The sample is incubated at 4° C. for at least 16 hours to precipitate the exosomes, followed by centrifugation at 10,000×g for 1 hour at 4° C. The supernatant is removed and the pellet is resuspended in 0.01 volume of PBS.

Exosome particle size and concentration are measured using Nanoparticle Tracking Analysis (NTA; Nanosight) and by ELISA. Exosome particles prepared from SM30, MEL2 and SK11 cells are in the expected size range of 88±2.9 nm. The concentration of exosomes bearing the exosome marker CD63 is measured by ELISA, using known concentrations of exosomes prepared from HT1080 human fibrosarcoma cells to prepare a standard curve. Samples (from SM30, MEL2 and HT1080 cells) are adsorbed to the ELISA plate by incubation overnight in PBS. The PBS is removed and wells are washed 3 times in wash buffer (Thermo Scientific) followed by incubation with primary mouse anti-CD63 antibody for 1 hour at room temperature. The primary antibody is removed followed by washing 3 times in wash buffer and incubation with secondary antibody (HRP conjugated anti-mouse) at 1:3,000 dilution for 1 hour at room temperature. The wells are washed 3 additional times with wash buffer and incubated in Super sensitive TMB ELISA substrate (Sigma, St. Louis, Mo.) for 0.5 hour followed by addition of ELISA stop solution (InVitrogen, Carlsbad, Calif.). The concentration of exosomes is determined by optical density in a standard plate reader at a wavelength of 450 nm.

The same methods can be used to prepare exosomes from chondrogenic precursor cells. Exosomes purified in this fashion can be used immediately or stored at −80° C. until needed.

### Example 5: Exosome-Containing Graft

Exosomes (fresh or thawed) are applied (optionally aseptically) to a biological carrier such as a collagen gel or sponge, or to a synthetic biomaterial, by dropwise application of exosome suspension to the support, followed by freeze-drying. Various ranges of exosome concentrations are used, e.g., from 1×10⁶ to 1×10⁹ exosomes/cc of biological or synthetic carrier. For a rat ectopic graft, carrier totaling about 0.5 cc is used. A total of 8×10⁵ to 1×10⁶ particles loaded onto the carrier by dropwise addition of 200 to 500 μl, depending on exosome particle concentration.

After application of the exosome suspension to the carrier, the exosome-loaded carrier is freeze-dried as described in Example 2. Following the freeze dry process, the exosome-loaded carrier is stored at room temperature or frozen.

For bone regeneration, an exosome-loaded carrier is placed in a surgically created muscle pouch in back of an adult rat; as described in Example 2, above. After 6 to 12 weeks, implants are recovered and bone formation is assessed using histological and biochemical characterization; e.g., as described in Example 2.

### Example 6: Conditioned Medium-Containing Graft

Conditioned medium (e.g., from osteogenic and/or chondrogenic precursor cells) can be used to prepare graft-forming units which contain a mixture of secreted factors including, but not limited to, exosomes. Conditioned medium is harvested from cells after osteogenic or chondrogenic induction as described above.

Conditioned medium can be concentrated prior to its application to a biological carrier. To obtain concentrated conditioned medium, 500 mL of conditioned medium from 10 T225 tissue culture flasks containing SM30 cells, grown as described above in Example 1, is introduced into a filtration cartridge with a molecular weight cut-off of 10 kd (preventing loss of most growth factors). The cartridge is then subjected to centrifugation to reduce the volume of medium to, e.g., 5 to 50 mL, generating a 10-100 fold concentration over starting material.

Conditioned medium, either concentrated or un-concentrated, is applied drop-wise to a collagen sponge and freeze-dried, as described above, prior to implantation.

### Example 7: Graft Containing Fractionated Conditioned Medium

5 mL of concentrated conditioned medium, obtained as described above in Example 7, is fractionated by HPLC. Specific HPLC fractions are applied, alone or in combination with other fractions, to a biological carrier as described above, prior to implantation.

### Example 8: Composite Graft

4D20.8, 7PEND24, 7SMOO32, or E15 cells are grown as monolayers and expanded using conditions described previously (Sternberg et al., Regen. Med., vol. 8, no. 2, pp. 125-144, 2013; U.S. Patent Application Publication No. 2014/0234964). After tissue culture expansion, the cells are dissociated (e.g., using trypsin-EDTA or Accutase) at 37° C. for 3-5 minutes and resuspended in chondrogenic differentiation medium consisting of DMEM (high glucose), penicillin/streptomycin (100 U/mL penicillin, 100 μg/mL streptomycin), GlutaMAX™ (2 mM), pyruvate (10 mM), dexamethasone (0.1 μM), L-proline (0.35 mM), 2-phospho-L-ascobic acid (0.17 mM), ITS (6.25 μg/mL transferrin, 6.25 ng/mL selenious acid, 1.25 mg/mL serum albumin and 5.35 μg/mL linoleic acid), plus 10 ng/mL TGF-β3 and either 10 ng/mL BMP-4 or 100 ng/mL GDF5. The cells are then seeded into a tissue culture dish, and maintained for a period varying from one week to 4 weeks. After chondrogenic differentiation, the cells, or cell-derived preparations derived therefrom, are loaded onto a biological carrier to form a cartilaginous layer. Prior to, or subsequent to, loading of the chondrogenic cells (or cell-derived preparation derived therefrom) onto the carrier, osteogenic cells (or cell-derived preparations derived therefrom) are loaded onto the same carrier to form an osteogenic layer.

1. A composition comprising:
(a) a cell-derived preparation from an osteogenic precursor cell, a chondrogenic precursor cell, or both; and
(b) a biological carrier.

2. The composition of claim 1, wherein the cell-derived preparation is selected from the group consisting of one or more of:
(a) a lyophilisate of an osteogenic precursor cell or a chondrogenic precursor cell;
(b) a lysate of an osteogenic precursor cell or a chondrogenic precursor cell;
(c) an extract of an osteogenic precursor cell or a chondrogenic precursor cell;
(d) an exosome suspension from an osteogenic precursor cell or a chondrogenic precursor cell; and
(e) conditioned medium from an osteogenic precursor cell or a chondrogenic precursor cell.

3. The composition of claim 1, wherein the osteogenic precursor cell and the chondrogenic precursor cell are not mesenchymal stem cells, are obtained by differentiation of a progenitor cell, and are not obtained from an embryoid body.

4. The composition of claim 3, wherein the progenitor cell comprises a clonal embryonic progenitor cell.

5. The composition of claim 1, wherein the osteogenic precursor cell is obtained by differentiation of a clonal progenitor cell line selected from the group consisting of a SM30, MEL2, SK11 and 4D20.8 or a progenitor cell that expresses one or more of the following markers: MMP1, MYL4, ZIC2, DIO2, DLK1, HAND2, SOX11, COL21A1, PTPRN and ZIC1.

6-7. (canceled)

8. The composition of claim 1 wherein the osteogenic precursor cells express one or more markers chosen from integrin-binding sialoprotein (TBSP), osteopontin (SPP1), alkaline phosphatase, tissue-nonspecific isozyme (ALPL), and BMP-2.

9-13. (canceled)

14. The composition of claim 1, wherein the chondrogenic precursor cell is obtained by differentiation of a clonal progenitor cell line selected from the group consisting of 4D20.8, 7PEND24, 7SMOO32 and E15 or a progenitor cell that expresses one or more of the following markers: DIO2, DLK1, FOXF1, GABRB1, COL21A1, and SRCRB4D.

15. (canceled)

16. The composition of claim 1, wherein the chondrogenic precursor cell expresses one or more markers chosen from collagen, type II, alpha 1 (COL2A1) and aggrecan (ACAN).

17. The composition of claim 1, wherein the biological carrier is a collagen, a collagen coated with a ceramic, a hydrogel, a hydrogel supplemented with a ceramic, a collagen sponge, a ceramic, a hydroxyapatite, a tricalcium phosphate, a collagen/ceramic composite, a hyaluronan, a fibrin, an elastin, a gelatin, a naturally-occurring extracellular matrix (ECM), MATRIGEL, an amnion, a demineralized bone matrix (DBM), a synthetic ECM, a polyglycolic acid (PGA), a polylactic acid (PLA), a polycaprolactone (PCL), or combinations thereof.

18-51. (canceled)

52. The composition of claim 1, for use in treating musculoskeletal conditions, osteochondritis dessicans (OCD), deep osteochondral joint defects of the knee or hip, bone or joint injury or trauma, a surgically created defect from an osteochondral graft harvesting procedure, for use in supplementing bone grafting spinal fusion procedures, orthopedic bone reconstruction, for use in promoting bone and/or cartilage formation, or for use in augmenting autologous bone grafting.

53. The composition of claim 2, wherein the composition comprises the lysate of an osteogenic precursor cell and is capable of extensive bone formation in vivo, as compared to growth medium.

54. A method for making a therapeutic composition for promoting bone formation, the method comprising:
(a) combining osteogenic precursor cells (OPCs) with a biological carrier; and
(b) performing one of the following:
(i) lyophilizing the combination of step (a); or
(ii) lysing the cells present on the biological carrier to generate a graft forming unit.

55. A method of making a therapeutic composition for promoting bone formation, the method comprising, combining one or more of a lysate from osteogenic precursor cells (OPCs), an extract from OPCs, exosomes from OPCs, and conditioned medium from a culture of OPCs with a biological carrier to generate a graft forming unit.

56. The method of claim 55, wherein the OPCs are differentiated from clonal embryonic progenitor cells, comprise clonal human cells, and are not obtained from an embryoid body.

57. The method of claim 56, wherein the osteogenic precursor cell is obtained by differentiation of a clonal progenitor cell line selected from the group consisting of a SM30, MEL2, SK11 and 4D20.8 or a progenitor cell that expresses one or more of the following markers: MMP1, MYL4, ZIC2, DIO2, DLK1, HAND2, SOX11, COL21A1, PTPRN and ZIC1.

58. The method of claim 55, wherein the graft forming unit is used for treating musculoskeletal conditions, osteochondritis dessicans (OCD), deep osteochondral joint defects of the knee or hip, bone or joint injury or trauma, a surgically created defect from an osteochondral graft harvesting procedure, for use in supplementing bone grafting spinal fusion procedures, orthopedic bone reconstruction, for use in promoting bone and/or cartilage formation, or for use in augmenting autologous bone grafting.

59. The method of claim 55, wherein the graft forming unit comprises the lysate of an osteogenic precursor cell and is capable of extensive bone formation in vivo, as compared to growth medium.

60. The method of claim 59, wherein the graft forming unit is capable of inducing bone formation by between about 3 to 6 weeks.

61. The method of claim 55, wherein the OPCs express one or more markers chosen from bone sialoprotein II (TBSP), osteopontin (SPP1) and alkaline phosphatase, tissue-nonspecific isozyme (ALPL).

62. The method of claim 55, wherein the biological carrier is a collagen, a collagen coated with a ceramic, a hydrogel, a hydrogel supplemented with a ceramic, a ceramic, a collagen sponge, a hydroxyapatite, a tricalcium phosphate, a collagen/ceramic composite, a hyaluronan, a fibrin, an elastin, a gelatin, a naturally-occurring extracellular matrix (ECM), MATRIGEL, an amnion, a demineralized bone matrix (DBM), a synthetic ECM, a polyglycolic acid (PGA), a polylactic acid (PLA), a polycaprolactone (PCL), or combinations thereof.
